(19) United States
(12) Patent Application Publication
(10) Pub. No.: US 2026/0364385 A1
(43) Pub. Date: Nov. 19, 2026

(54) MONITORING AND CONTROL OF HISTOTRIPSY PROCEDURES

(71) Applicant: INSIGHTEC LTD., Tirat Carmel (IL)

(72) Inventors: Netanel NAFTALIS, Or Akiva (IL); Yoav LEVY, Hinanit (IL); Israel SCHUSTER, Kyriat Tivon (IL); Oleg PRUS

(21) Appl. No.: 19/470,591

(22) PCT Filed: Apr. 2, 2024
(86) PCT No.: PCT/IB2024/053195

§ 371 Date: Sep. 29, 2025

Related U.S. Application Data
(60) Provisional application No. 63/492,784, filed on Mar. 29, 2023.

(51) Int. Cl. A61N 7/00

(52) U.S. Cl. CPC A61N 7/00; A61N 2007/0078; A61N 2007/0095

(57) ABSTRACT

A histotripsy procedure controllably causes tissue destruction in a target internal anatomic region by: sonicating a target region with a series of acoustic pulses; detecting ultrasound reflection signals from the target region; controlling an ultrasound transducer to deliver, to the target region, a sequence of acoustic pulses with sufficient amplitude to induce sufficient cavitation to mechanically rupture tissue therein; receiving data characterizing the detected reflection signals; based on the data, computationally estimating a level or a location of acoustic activity or an acoustic field distribution at the target region; and based on a plurality of acoustic activity level estimations or acoustic field distribution estimations: stopping delivery of the acoustic pulses; continuing delivery of the acoustic pulses at a constant power level; increasing a power level for delivery of the acoustic pulses; or decreasing a power level for delivery of the acoustic pulses.

### TECHNICAL FIELD

The present disclosure relates to systems and methods for monitoring and controlling histotripsy procedures in focused ultrasound treatments.

### BACKGROUND

Histotripsy involves the delivery of acoustic energy in the form of short (generally less than 50 μsec), high-amplitude acoustic pulses that deliver relatively low total energy at high peak pressure. This induces brief cavitation to mechanically rupture targeted tissue. Cavitation occurs when a sufficiently negative pressure is applied to tissue to cause microbubble formation from fluid vaporization and release of dissolved gas. Once formed, the microbubbles exhibit highly dynamic patterns of oscillation and inertial collapse that produce cellular and tissue disruption. Whereas lesioning applications such as those involving high-intensity focused ultrasound are sensitive to the amount of energy reaching the target, histotripsy is more sensitive to the negative peak pressure. The required pulse intensity for inducing cavitation is very high (>20 MPa).

Of course, all treatments involving tissue ablation must be monitored and controlled to ensure patient safety and treatment effectiveness. Histotripsy initiation is mainly a threshold (rather than a cumulative) effect, meaning that the onset of the effect is sudden as a treatment parameter, typically delivered power, is increased. Once the power level increases past a threshold, the extent of the histotripsy effect grows significantly with the power and/or number of repetitions. Consequently, any increase in delivered power may trigger the effect with no warning signs. At the high-power levels required to induce cavitation, the risk of injury due to excessive energy deposit (i.e., more than is necessary to induce cavitation at levels necessary for treatment) is significant. Therefore, fast feedback is crucial to allow histotripsy-based treatments to be efficient and safe.

Achieving precise, real-time intracorporeal imaging to detect the onset and extent of cavitation remains a challenge. Standard active ultrasound imaging is fast but limited in resolution, particularly in transcranial procedures where the skull produces aberrations that are different for each patient. An alternative is magnetic resonance imaging (MRI), which provides better images and is less subject to interference from bony structures. However, MRI is a relatively slow technique, so it is difficult to update images over short enough intervals to permit fine control over histotripsy procedures. Moreover, even if the onset of cavitation can be detected quickly and reliably, the optimal post-detection control sequence is not self-evident. If power is reduced or cut off too quickly, the desired treatment effect will not be attained; if power is maintained at the current level, cavitation may diminish below therapeutically useful levels; and increasing power risks injury to the patient.

### SUMMARY

The present disclosure provides systems and methods for monitoring and controlling histotripsy procedures in focused ultrasound treatments.

In one aspect, a system for controllably causing tissue destruction in a target internal anatomic region is described. The system includes: an ultrasound transducer for sonicating a target region with a series of acoustic pulses; an acoustic detection system comprising at least one detector for detecting ultrasound reflection signals from the target region following at least some of the acoustic pulses; and a controller. The controller is configured to control the ultrasound transducer to deliver, to the target region, a sequence of acoustic pulses each having a duration no greater than 100 μsec with sufficient amplitude to induce sufficient cavitation in the target region to mechanically rupture tissue therein; and receive, from the acoustic detection system, data characterizing the detected reflection signals. Based at least on the data characterizing the detected reflection signals, the controller is configured to estimate a level or a location of acoustic activity or an acoustic field distribution at the target region. Based on a plurality of acoustic activity level estimations or acoustic field distribution estimations, the controller is configured to stop delivery of the acoustic pulses, continue delivery of the acoustic pulses at a constant power level, increase a power level for delivery of the acoustic pulses, or decrease a power level for delivery of the acoustic pulses.

In another aspect, a method of controllably causing tissue destruction in a target internal anatomic region is described. The method includes: sonicating a target region with a series of acoustic pulses; detecting ultrasound reflection signals from the target region following at least some of the acoustic pulses; controlling an ultrasound transducer to deliver, to the target region, a sequence of acoustic pulses each having a duration no greater than 100 μsec with sufficient amplitude to induce sufficient cavitation in the target region to mechanically rupture tissue therein; and receiving data characterizing the detected reflection signals. Based at least on the data characterizing the detected reflection signals, the method further includes computationally estimating a level or a location of acoustic activity or an acoustic field distribution at the target region. Based on a plurality of acoustic activity level estimations or acoustic field distribution estimations, the method further includes stopping delivery of the acoustic pulses; continuing delivery of the acoustic pulses at a constant power level; increasing a power level for delivery of the acoustic pulses; or decreasing a power level for delivery of the acoustic pulses.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of this disclosure. In the following description, various embodiments of the present disclosure are described with reference to the following drawings, in which:

FIG. 1A schematically depicts an exemplary ultrasound system in accordance with various embodiments of the current disclosure.

FIG. 1B schematically depicts an exemplary MRI system in accordance with various embodiments of the current disclosure.

FIG. 2 depicts an implementation of an acoustic reflector substantially close to a target region in accordance with some embodiments.

FIG. 3 is a graph depicting an example sonication event, in which applied acoustic power levels are selected according to reflection signals caused by histotripsy events, in accordance with some embodiments.

FIG. 4 is a flow chart illustrating an exemplary approach for monitoring and controlling histotripsy procedures in focused ultrasound treatments in accordance with some embodiments.

### DETAILED DESCRIPTION

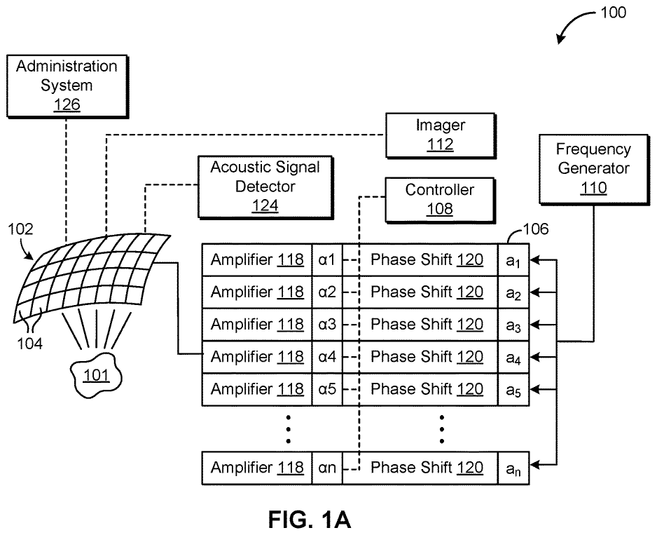

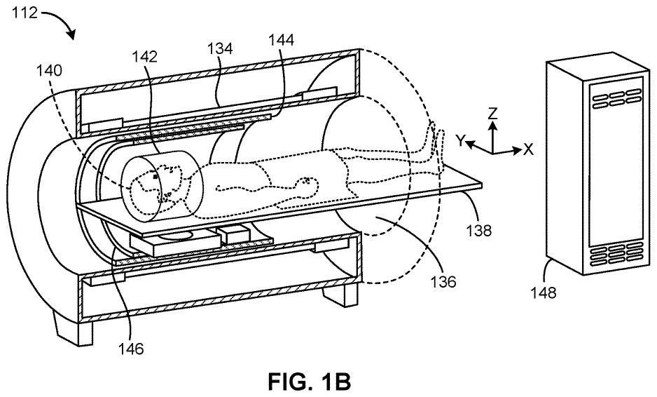

FIG. 1A illustrates an exemplary ultrasound system 100 for generating and delivering a focused acoustic energy beam to a target region 101 within a patient's body. The illustrated system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) or other contoured shape suitable for placement on the surface of the patient's body, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50 Ω, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each including or consisting of an amplifier 118 and a phase delay circuit 120; each drive circuit drives one of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual transducer elements 104 of the transducer array 102 at the same frequency, but at different phases and/or different amplitudes.

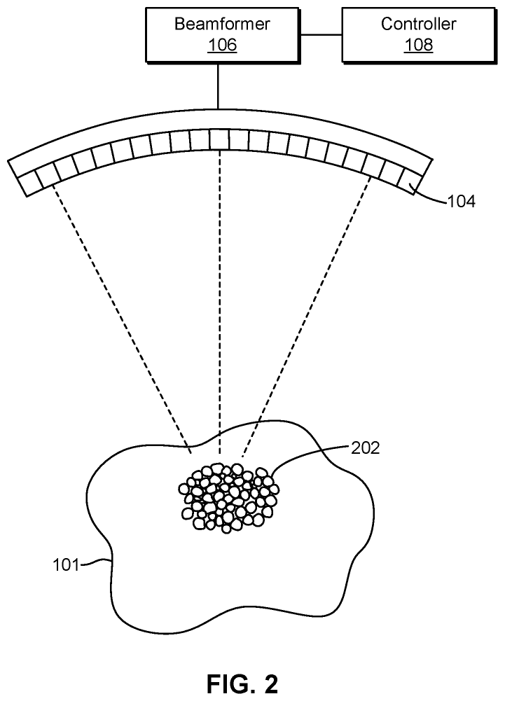

The amplification or attenuation factors ai-an and the phase shifts a₁-an imposed by the beamformer 106 serve to transmit and focus ultrasonic energy through the intervening tissue located between the transducer elements 104 and the target region onto the target region 101, and account for wave distortions induced in the intervening tissue. The amplification factors and phase shifts are computed using the controller 108, which may provide the computational functions through software, hardware, firmware, hardwiring, or any combination thereof. In various embodiments, the controller 108 utilizes a general-purpose or special-purpose digital data processor programmed with software in a conventional manner, and without undue experimentation, to determine the frequency, phase shifts and/or amplification factors necessary to obtain a desired focus or any other desired spatial field patterns at the target region 101. In certain embodiments, the computation is based on detailed information about the characteristics (e.g., the type, size, location, property, structure, thickness, density, structure, etc.) of the intervening tissue located between the transducer element 104 and the target and their effects on propagation of acoustic energy. Such information may be obtained from an imager 112. The imager 112 may be, for example, a magnetic resonance imaging (MRI) device, a computer tomography (CT) device, a positron emission tomography (PET) device, a single-photon emission computed tomography (SPECT) device, or an ultrasonography device. Image acquisition may be three-dimensional (3D) or, alternatively, the imager 112 may provide a set of two-dimensional (2D) images suitable for reconstructing a three-dimensional image of the target region 101 and/or other regions (e.g., the region surrounding the target 101 or another target region). Image-manipulation functionality may be implemented in the imager 112, in the controller 108, or in a separate device. In addition, the ultrasound system 100 and/or imager 112 may be utilized to detect signals from an acoustic reflector (e.g., microbubbles 202, see FIG. 2) located substantially close to the target region 101 as further described below. Additionally or alternatively, the system 100 may include an acoustic-signal detection device (such as a hydrophone or suitable alternative) 124 that detects transmitted or reflected ultrasound from the acoustic reflector, and which may provide the signals it receives to the controller 108 for further processing. In addition, the ultrasound system 100 may include an administration system 126 for parenterally introducing the acoustic reflector into the patient's body. The imager 112, the acoustic-signal detection device 124, and/or the administration system 126 may be operated using the same controller 108 that facilitates the transducer operation; alternatively, they may be separately controlled by one or more separate controllers intercommunicating with one another.

FIG. 1B illustrates an exemplary imager-namely, an MRI apparatus 112. The apparatus 112 may include a cylindrical electromagnet 134, which generates the requisite static magnetic field within a bore 136 of the electromagnet 134. During medical procedures, a patient is placed inside the bore 136 on a movable support table 138. A region of interest 140 within the patient (e.g., the patient's head) may be positioned within an imaging region 142 wherein the electromagnet 134 generates a substantially homogeneous field. A set of cylindrical magnetic field gradient coils 144 may also be provided within the bore 136 and surrounding the patient. The gradient coils 144 generate magnetic field gradients of predetermined magnitudes, at predetermined times, and in three mutually orthogonal directions. With the field gradients, different spatial locations can be associated with different precession frequencies, thereby giving a magnetic-resonance (MR) image its spatial resolution. An RF transmitter coil 146 surrounding the imaging region 142 emits RF pulses into the imaging region 142 to cause the patient's tissues to emit MR response signals. Raw MR response signals are sensed by the RF coil 146 and passed to an MR controller 148 that then computes an MR image, which may be displayed to the user. Alternatively, separate MR transmitter and receiver coils may be used. Images acquired using the MRI apparatus 112 may provide radiologists and physicians with a visual contrast between different tissues and detailed internal views of a patient's anatomy that cannot be visualized with conventional x-ray technology.

The MRI controller 148 may control the pulse sequence, i.e., the relative timing and strengths of the magnetic field gradients and the RF excitation pulses and response detection periods. The MR response signals are amplified, conditioned, and digitized into raw data using a conventional image-processing system, and further transformed into arrays of image data by methods known to those of ordinary skill in the art. Based on the image data, the target region (e.g., a tumor or a target BBB) can be identified.

To perform targeted drug delivery or tumor ablation, it is necessary to determine the location of the target region 101 with high precision. Accordingly, in various embodiments, the imager 112 is first activated to acquire images of the target region 101 and/or non-target region (e.g., the healthy tissue surrounding the target region, the intervening tissue located between the transducer array 102 and the target region 101 and/or any regions located near the target) and, based thereon, determine anatomical characteristics (e.g., the tissue type, location, size, thickness, density, structure, shape, vascularization) associated therewith. For example, a tissue volume may be represented as a 3D set of voxels based on a 3D image or a series of 2D image slices and may include the target region 101 and/or non-target region.

To create a high-quality focus at the target region 101, it may be necessary to calibrate the transducer elements 104 and take into account transducer geometric imperfections resulting from, for example, movement, shifts and/or deformation of the transducer elements 104 from their expected locations. In addition, because the ultrasound waves may be scattered, absorbed, reflected and/or refracted when traveling through inhomogeneous intervening tissues located between the transducer elements 104 and the target region 101, accounting for these wave distortions may also be necessary in order to improve the focusing properties at the target region 101.

Referring to FIG. 2, ultrasound waves transmitted from all (or at least some) transducer elements 104 are reflected by the acoustic reflectors 202. The acoustic reflectors 202 may consist essentially of microbubbles generated by the ultrasound waves and/or introduced parenterally by an administration system. In some embodiments, the administration system 126 introduces a seed microbubble into the target region 101; the transducer 102 is then activated to transmit ultrasound waves to the seed microbubble for generating a cloud of microbubbles. Approaches to generating the microbubbles and/or introducing the microbubbles to the target region 101 are provided, for example, in PCT Publication No. WO2018/020315, PCT Application Nos. PCT/US2018/064058 (filed on Dec. 5, 2018), PCT/IB2018/001103 (filed on Aug. 14, 2018), PCT/US2018/064892 (filed on Dec. 11, 2018), PCT/IB2018/000841 (filed on Jun. 29, 2018), and PCT/US2018/064066 (filed on Dec. 5, 2018), U.S. Patent Publication No. 2019/0083065, and U.S. patent application Ser. No. 15/837,392 (filed on Dec. 11, 2017), the contents of which are incorporated herein by reference.

In accordance with various embodiments, control of histotripsy procedures is based on spatial acoustic imaging of the target region 101. This provides, in essence, a 3D picture of the acoustic energy distribution, enabling detection of high-energy events, such as cavitation, which stand out from the background. The acoustic energy is either generated by the histotripsy events (e.g., sound waves emitted by the cavitating bubbles) or originated by the transducer transmission and reflected by the products of the histotripsy events (e.g., sound waves transmitted from the transducer and reflected by the cavitating bubbles) during or after the events. As used herein, the acoustic imaging terms “reflected signals” and “reflection signals” refer to signals generated by the acoustic energy regardless of the source of this energy. Control of applied acoustic power may be based on a plurality of successive cavitation level estimations, which may result in stopping delivery of acoustic pulses altogether, continuing the pulse sequence at a constant power level, or increasing the delivered power at a rate determined at least in part by the successive cavitation level estimations. For example, delivered power may be increased until an observed acoustic event (i.e., a significant confined reflection signal)—which likely indicates cavitation—is observed at a focal region within the target region. At this point, power may be increased slowly, usually, increase of two to five percent in respect to prior acoustic power. After several events have been observed at the focal region, usually, one to four events per focal point, the treatment power may be held constant until several more (e.g., one to ten) events are observed, at which point treatment is complete for the focal region. Now the power may be increased or decreased by a factor (e.g., 40%), the focal region shifted to a new portion of the target region, and the foregoing procedure repeated. If no events are detected after power has been increased to a maximum safe level, the focal point may be shifted to another target region, or treatment may be stopped altogether. The reflection signals received from the current focal region may also be used to improve focusing on points nearby.

In various embodiments, a particular frequency or band of frequencies of a reflection signal is selected for analysis. The reflection signal may be analyzed using the first harmonic of the transmission signal (i.e., the same frequency as the transmitted signal). Additionally or alternatively, the reflection signal may be analyzed using any other frequency band (e.g., second harmonic frequency, sub harmonic frequency, and/or ultra-harmonic frequency). In some embodiments, a wide band signal is used for analysis. For the measurement itself, a single hydrophone or an array of hydrophones may be employed. Without loss of generality, the following description assumes that reflection signals are detected using a hydrophone array. As the difference between two reflected signals through the skull is monitored, effects due to aberration and attenuation are eliminated, and system calibration is generally unnecessary to obtain quantitative results.

Using the short pulses characteristic of histotripsy with a hydrophone array enables generation of 3D acoustic activity maps of the target area, a procedure also known as passive acoustic mapping or PAM. This may be accomplished by constructing a 3D image of the spatial acoustic field using reflected signals from the spatially distributed transducer elements. The distance between each transducer element and each voxel in the spatial region of interest is known, as is the speed of sound through the relevant tissue; accordingly, based on the time of flight and/or the phase delay and by aggregating measurements from multiple sensing transducer elements to resolve degeneracies, the response to an event at each voxel of interest can be computed. As a result, the analysis methods described above can be implemented on a per-voxel basis in the region of interest, providing spatial information relating to treatment progress, efficacy, and safety. For example, excessive activity may be identified in an undesired location (e.g., unintended shift of the main beam effect from the target area).

Usually, the signals reflected from the histotripsy event are mixed with signals from other reflectors in the body such as bones. In some embodiments, generating a cavitation activity map for each pulse in the sequence facilitates comparison between two pulses (e.g., the first and second pulses in the sequence) and thus generation of a 3D map of the acoustic field generated as a response to the transmitted pulse in the region of interest. These maps can be overlayed on MRI maps to estimate the treatment outcome, with regard to both efficacy and safety.

In some embodiments, the general pipeline for generation of an acoustic map for the controller includes: (a) generating a histotripsy event (e.g., cavitating bubbles); (b) receiving signals (e.g., emitted by or reflected off the cavitating bubbles), (c) optionally removing undesired parts of the signals (e.g., reflection of the transmitted treatment signals reflected by static anatomy like the skull), (d) correcting the signals for aberrations, and (e) summing the signals (e.g. field reconstruction).

Generating the histotripsy in the region of treatment may require pulses comprising as few as one cycle each. Even so, in some scenarios, pulses used to generate the acoustic map may be longer (e.g., longer than 3 cycles), which causes the resolution to decrease. To overcome this challenge, the reflection signals may be processed as follows:

First, for each receiver, two successive received signals are subtracted to remove the background. Then, a significant portion of the signal (on the order of the pulse length) is filtered for a specific frequency (usually the transmission frequency, or other frequency as discussed above), and the phase and amplitude of this signal portion are obtained at the selected frequency. Using a relatively long signal portion exploits more energy for the measurement and improves SNR by scarifying the resolution (assuming sparsity effects). This produces, for each receiver, a complex signal value (including phase, amplitude, or both) for the selected frequency.

To correct for wave propagation time and/or aberration, a phase delay correction is applied to the complex signal based on (i) the distance from the receiver to each reconstructed spatial point and/or (ii) the anatomic related aberrations.

The wave propagation time is the time of flight of the acoustic wave from the histotripsy event to each receiver based on the velocity of the sound in the water and in watery tissue. The correction from the wave propagation time is achieved by a simple calculation based of lengths and velocities.

The correction for aberrations is a correction for the aberration caused by more complex tissues such as the skull. The correction for the aberration can be done using: (i) a physical model, such as described in U.S. Pat. No. 10,765,892, the entire disclosure of which is hereby incorporated by reference; (ii) intraoperative measurements, such as those described in U.S. Patent Publ. No. 2019/0308038, the entire disclosure of which is hereby incorporated by reference; and/or (iii) other models, such as those described in U.S. Pat. No. 11,291,430, the entire disclosure of which is hereby incorporated by reference.

The corrected complex signal values from all receivers are summed to obtain the amplitude (corresponding to the magnitude of the acoustic field) at each reconstructed point. The phase is also obtained for each point. The phase and amplitude values over all reconstructed points constitute the 3D PAM.

Another approach to 3D spatial reconstruction is to reconstruct a single 2D plane point by point within the target and use an angular spectrum method to create adjacent planes and, hence, a 3D spatial map. Specifically, the angular spectrum method may comprise expanding a complex wave field into a summation of plane waves of the same frequency and different directions. The technique can predict an acoustic pressure field distribution over a plane, based upon knowledge of the pressure field distribution at a parallel plane.

The following paragraphs present the results of a typical histotripsy experiment using the histotripsy controller. In each trigger initiated by the controller, the transducer sonicates a sequence of two consecutive pulses, each of ~50 us length. Every sonication sequence triggers the acquisition system to acquire the acoustic signal from the treatment region of interest. The acoustic signal is then analyzed as described above (with reference to reflection signals), and the 3D acoustic map is delivered to the histotripsy controller to decide the next trigger parameters. For this treatment, the histotripsy activity was monitored via the maximum intensity of the reconstructed acoustic map.

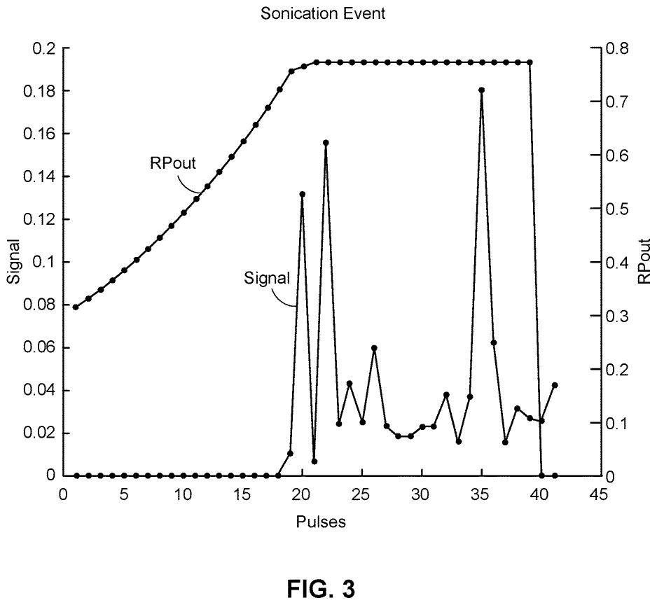

The left axis in FIG. 3 plots the maximum intensity in the treatment region measured in arbitrary units, marked as “Signal.” According to previous experiments, a Signal higher than 0.015 was identified as evidence for histotripsy; therefore, Signal=0.015 was set to be the histotripsy treatment threshold.

The spatial distribution of the field in the 3D acoustic map can be analyzed. When the power is lower than the histotripsy event generation threshold, the distribution of the measured acoustic field is wide. When the power lever exceeds the histotripsy threshold level, the localized histotripsy event generates a strong field with narrow distribution (e.g., >50% of the energy within a 3 mm radius). In addition, if the power level is above the safety threshold, the histotripsy effect might be bigger or might affect a bigger area and therefore, the reconstructed acoustic field might have a wider distribution. In some embodiments, the acoustic field distribution is used for determining the treatment threshold and/or the safety threshold.

In this experiment, the treatment was managed using basic tools. Specifically, the measured maximum acoustic intensity (Signal in FIG. 3) was the input, and the output voltage (RPout in FIG. 3) was controlled as the output, which in turn increases FUS power. The starting output voltage was 30% from the maximum voltage allowed by the user. If the Signal was higher than the threshold, than a counter for “histotripsy events” was increased by one, i.e., events=events+1. If “events” was 0, the next trigger was set to increase its voltage by 5%. If “events” was bigger than 0 and less than 3, the next trigger was set to increase its voltage by 2%. If “events” was greater than 2 but smaller than 20, the next trigger was set as the previous voltage, until reaching the desired accumulated events, which was set in this experiment to “events” equal to 20. If “events” was higher than 20, the next trigger was set zero, or in other words, the treatment was ended. The right-axis in FIG. 3 plots the voltage applied (corresponding to the delivered power) according to the aforementioned controller algorithm.

In addition to or as an alternative to the hardware platform described above with reference to FIGS. 1A-2, a representative hardware platform for implementation of the present invention is described in U.S. Pat. No. 11,918,832, the entire disclosure of which is hereby incorporated by reference. The hardware system may include an imaging device (e.g., a magnetic resonance imaging (MRI) device) to characterize tissue types and/or properties of the target region and/or its surrounding tissue; each type and location of tissue, depending on its properties, may have corresponding tolerances for histotripsy; accordingly, the imaging device may be used to spatially characterize tissue tolerance, and this spatial representation may be used for comparison to a spatial acoustic map. The '832 patent also describes suitable ultrasound transducer arrangements and driver circuitry (similar to those described above with reference to FIGS. 1A-2).

A controlled histotripsy treatment of a single point might cover a volume of 0.001 to 1 cubic centimeter (cc), and a typical ablated volume can be about 0.1 cc. Targets to be treated by histotripsy treatment are typically 0.5 cc up to (but not limited to) 125 cc. The careful treatment control that starts with power below the treatment threshold and increased gradually (as described above with reference to FIG. 3) is a good starting point for a volumetric treatment. In addition, this careful threshold detection gives high safety margin. However, repeating this process for each point to be ablated within the target may take too long since the target might be comprised of thousands of points that need to be ablated.

Therefore, there is a need for a strategy for fast ablation of each point in the target while preserving the efficacy and safety of the treatment. In experiments, it was determined that variation in the histotripsy threshold is very high; however, the threshold of two close points within a single tissue type is not that different (up to 50% difference). Therefore, in some embodiments, once a threshold for a specific point is measured by the controller, the controller can set a working power level for an area and treat the entire area with the same power level. The working power for an area might be a bit higher (10%-100%) than the threshold that was found for a point in that area. In some embodiments, the area is defined as all points at the distance smaller than a predefined radius from the point for which the threshold was measured. A typical predefined radius might be 10-20 mm. In some embodiments, a medical image of the treated area is used to segment the area by tissue type and the working power selection might be limited to tissues from the same type. In some embodiments, the histotripsy events in the treated area are used for gradual correction of the focusing. In some embodiments, the treatment of the points around the point in which the threshold was measured is monitored by the controller in the same way that the treatment of the first point was monitored to assure efficacy and safety. If the signal of cavitation events is lost, the controller might start increasing the treatment power. If the reflection from the histotripsy events raises safety concerns, the controller might reduce the power. In some embodiments, the treatment area for which the working power is applicable is not limited to a specific radius. Treatment may continue as long as efficacy and safety are tolerable, and the power level may be adjusted when needed.

In some embodiments, the system has also an imaging device used for guiding the targeting. In some embodiments, the imaging device is an MRI, a CT, and/or an ultrasound scanner. In order to assure treatment in the desired place, images may be taken and the location of the actual interaction of the acoustic beam and the tissue (actual place of the treatment) may be located on the images. Images can be taken before the histotripsy activity, during the histotripsy activity, and/or in between the histotripsy events. In some embodiments, images are thermal images of acoustic heating by the acoustic beam. Thermal images can be produced using MRI thermometry or ultrasound thermometry. The heating is typically mild temporal heating that has no significant clinical effect. In some embodiments, the images show tiny blood cell extravasation caused by explosions of acoustic contrast agents or (usually a small number of weak) histotripsy events. Blood cell extravasation can be imaged by MRI (T2* and others) or CT imaging. In some embodiments, ultrasound imaging is used to detect the bubbles generated by the histotripsy. In some embodiments, the controller automatically locates the actual place of the treatment on the image. In some embodiments, the controller adjusts the treatment parameters to shift the actual place of the treatment towards the desired place of the treatment. In some embodiments, the controller configures or triggers the imaging in the right place and/or time.

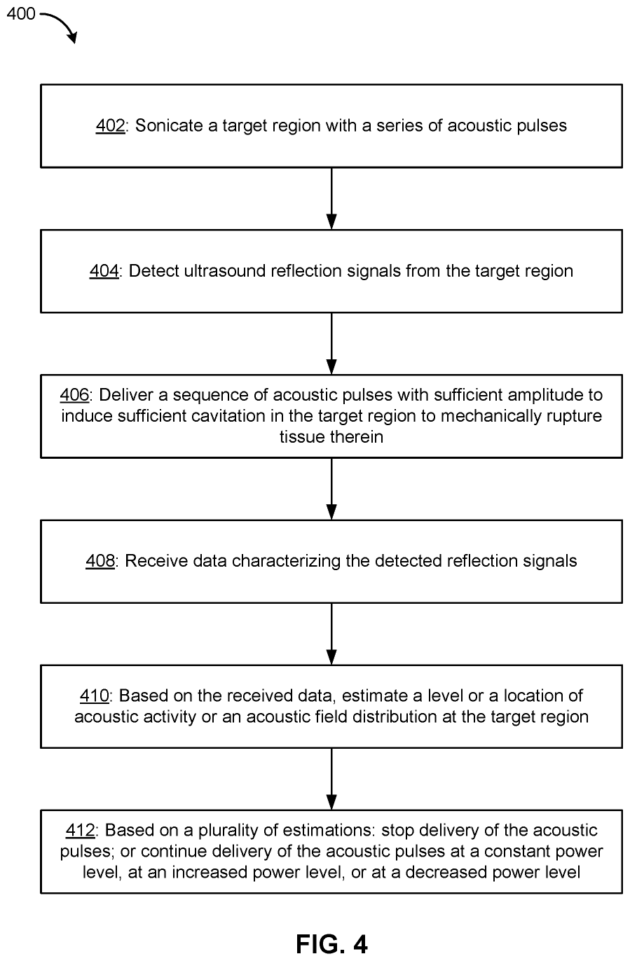

FIG. 4 is a flow diagram illustrating an example process 400 for controllably causing tissue destruction in a target internal anatomic region in accordance with some implementations. The process may be governed by instructions that are stored in a computer memory or non-transitory computer readable storage medium. The instructions may be included in one or more programs stored in the non-transitory computer readable storage medium. When executed by one or more processors (e.g., 108 and/or 148), the instructions cause the system to perform the process. The non-transitory computer readable storage medium may include one or more solid state storage devices (e.g., Flash memory), magnetic or optical disk storage devices, or other non-volatile memory devices. The instructions may include source code, assembly language code, object code, or any other instruction format that can be interpreted by one or more processors. Some operations in the process may be combined, and the order of some operations may be changed.

In operation 402, an ultrasound system (e.g., 100) sonicates a target region (e.g., 101) with a series of acoustic pulses.

In operation 404, the system detects (e.g., 112) ultrasound reflection signals from the target region following at least some of the acoustic pulses.

In operation 406, the system controls an ultrasound transducer (e.g., 102) to deliver, to the target region, a sequence of acoustic pulses each having a duration no greater than 70 μsec, or no greater than 100 μsec (or having a duration outside of these ranges depending on the application), with sufficient amplitude to induce sufficient cavitation in the target region to mechanically rupture tissue therein.

In operation 408 the system receives data (e.g., 124) characterizing the detected reflection signals.

In operation 410, based at least on the data characterizing the detected reflection signals, the system computationally estimates a level or a location of acoustic activity or an acoustic field distribution at the target region.

In operation 412, based on a plurality of acoustic activity level estimations or acoustic field distribution estimations, the system stops delivery of the acoustic pulses (e.g., after pulse 39 in FIG. 3), continues delivery of the acoustic pulses at a constant power level (e.g., between pulses 21 and 39 in FIG. 3), increases a power level for delivery of the acoustic pulses (e.g., between pulses 1 and 20 in FIG. 3); or decreases a power level for delivery of the acoustic pulses. In some implementations, the plurality of acoustic activity level estimations or acoustic field distribution estimations comprises a plurality of successive acoustic activity level estimations or acoustic field distribution estimations.

In some implementations, detecting the ultrasound reflection signals includes use of a plurality of spatially distributed acoustic detectors.

In some implementations, the level of acoustic activity or the acoustic field distribution is determined at least in part based on locations of the acoustic detectors with respect to the target region.

In some implementations, the level of acoustic activity or the acoustic field distribution is determined at a plurality of spatially distributed locations.

In some implementations, the process further comprises computing a volumetric acoustic field in the target region by field reconstruction.

In some implementations, the acoustic pulses have a period in a range of 10 μs to 100 μs.

In some implementations, the process further comprises estimating the acoustic field distribution for a transmission frequency field, a second harmonic field, a sub harmonic field, and/or an ultra-harmonic field.

In some implementations, the process further comprises determining a treatment threshold and/or a safety threshold based at least in part on the acoustic field distribution.

In some implementations, the process further comprises, based on the plurality of acoustic activity level estimations, increasing the power level for delivery of the acoustic pulses, including increasing the power level at a rate determined at least in part by the acoustic activity level estimations.

In some implementations, the process further comprises setting the constant power level, the increased power level, or the decreased power level as a working power level for a plurality of points within the target area; and continuing delivery of the acoustic pulses at the working power level to the plurality of points within the target area without re-estimating the level of acoustic activity or the acoustic field distribution.

In some implementations, the process further comprises defining an area including the plurality of points as: all points at a distance smaller than a predefined radius from an initial point at which the level of acoustic activity or the acoustic field distribution was estimated; or all points in a segment of the target region having a tissue type corresponding to the tissue type at the initial point at which the level of acoustic activity or the acoustic field distribution was estimated.

In some implementations, the process further comprises acquiring images using an imaging device comprising a magnetic resonance imaging (MRI) device, a computer tomography (CT) device, or an ultrasound device; determining a location of an interaction of the acoustic pulses using the images acquired by the imaging device; and adjusting one or more treatment parameters to shift the location of the interaction to a desired location in the target region.

In some implementations, the images are thermal images of acoustic heating caused by the acoustic pulses, or the images are images sensitive to blood cell extravasation.

In some implementations, determining the location of the interaction comprises locating blood cell extravasation in the images, or otherwise determining the location of the interaction in the images.

More generally, functionality for performing controlled histotripsy at a target region may be structured in one or more modules implemented in hardware, software, or a combination of both. For embodiments in which the functions are provided as one or more software programs, the programs may be written in any of a number of high-level languages such as PYTHON, FORTRAN, PASCAL, JAVA, C, C++, C #, BASIC, various scripting languages, and/or HTML. Additionally, the software can be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

Reference have been made in detail to various implementations, examples of which are illustrated in the accompanying drawings. In the above detailed description, numerous specific details are set forth in order to provide a thorough understanding of the invention and the described implementations. However, the invention may be practiced without these specific details. In other instances, well-known methods, procedures, components, and circuits have not been described in detail so as not to unnecessarily obscure aspects of the implementations.

It will be understood that, although the terms “first,” “second,” etc. may be used herein to describe various elements, these elements should not be limited by these terms. These terms are only used to distinguish one element from another. For example, a first device could be termed a second device, and, similarly, a second device could be termed a first device, without changing the meaning of the description, so long as all occurrences of the first device are renamed consistently and all occurrences of the second device are renamed consistently. The first device and the second device are both devices, but they are not the same device.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. For example, “A, B, and/or C” means: A only; B only; C only; A and B; A and C; B and C; or A, B, and C. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

1. A system for controllably causing tissue destruction in a target internal anatomic region, the system comprising:
an ultrasound transducer for sonicating a target region with a series of acoustic pulses;
an acoustic detection system comprising at least one detector for detecting ultrasound reflection signals from the target region following at least some of the acoustic pulses; and
a controller configured to:
control the ultrasound transducer to deliver, to the target region, a sequence of acoustic pulses each having a duration no greater than 100 μsec with sufficient amplitude to induce sufficient cavitation in the target region to mechanically rupture tissue therein;
receive, from the acoustic detection system, data characterizing the detected reflection signals;
based at least on the data characterizing the detected reflection signals, estimate a level or a location of acoustic activity or an acoustic field distribution at the target region; and
based on a plurality of acoustic activity level estimations or acoustic field distribution estimations:
stop delivery of the acoustic pulses;
continue delivery of the acoustic pulses at a constant power level;
increase a power level for delivery of the acoustic pulses; or
decrease a power level for delivery of the acoustic pulses.

2. The system of claim 1, wherein:
the acoustic detection system comprises a plurality of spatially distributed acoustic detectors; and
the level of acoustic activity or the acoustic field distribution is determined at least in part based on locations of the acoustic detectors with respect to the target region.

3. The system of claim 1, wherein the level of acoustic activity or the acoustic field distribution is determined at a plurality of spatially distributed locations.

4. The system of claim 1, wherein:
the controller is configured to compute a volumetric acoustic field in the target region by field reconstruction; and
the acoustic pulses have a period in a range of 10 μs to 100 μs.

5. The system of claim 1, wherein the controller is configured to:
estimate the acoustic field distribution for a transmission frequency field, a second harmonic field, a sub harmonic field, and/or an ultra-harmonic field; and
determine a treatment threshold and/or a safety threshold based at least in part on the acoustic field distribution.

6. The system of claim 1, wherein the controller is configured to, based on the plurality of acoustic activity level estimations, increase the power level for delivery of the acoustic pulses, including increasing the power level at a rate determined at least in part by the acoustic activity level estimations.

7. The system of claim 1, wherein the controller is configured to:
set the constant power level, the increased power level, or the decreased power level as a working power level for a plurality of points within the target area; and
continue delivery of the acoustic pulses at the working power level to the plurality of points within the target area without re-estimating the level of acoustic activity or the acoustic field distribution.

8. The system of claim 7, wherein the controller is configured to define an area including the plurality of points as:
all points at a distance smaller than a predefined radius from an initial point at which the level of acoustic activity or the acoustic field distribution was estimated; or
all points in a segment of the target region having a tissue type corresponding to the tissue type at the initial point at which the level of acoustic activity or the acoustic field distribution was estimated.

9. The system of claim 1, further comprising an imaging device comprising a magnetic resonance imaging (MRI) device, a computer tomography (CT) device, or an ultrasound device;
wherein the controller is further configured to:
determine a location of an interaction of the acoustic pulses using images acquired by the imaging device; and
adjust one or more treatment parameters to shift the location of the interaction to a desired location in the target region.

10. The system of claim 9, wherein:
the images are thermal images of acoustic heating caused by the acoustic pulses, or the images are images sensitive to blood cell extravasation; and
the controller is configured to determine the location of the interaction in the images.

11. A method of controllably causing tissue destruction in a target internal anatomic region, the method comprising:
sonicating a target region with a series of acoustic pulses;
detecting ultrasound reflection signals from the target region following at least some of the acoustic pulses;
controlling an ultrasound transducer to deliver, to the target region, a sequence of acoustic pulses each having a duration no greater than 100 μsec with sufficient amplitude to induce sufficient cavitation in the target region to mechanically rupture tissue therein;
receiving data characterizing the detected reflection signals;
based at least on the data characterizing the detected reflection signals, computationally estimating a level or a location of acoustic activity or an acoustic field distribution at the target region; and
based on a plurality of acoustic activity level estimations or acoustic field distribution estimations:
stopping delivery of the acoustic pulses;
continuing delivery of the acoustic pulses at a constant power level;
increasing a power level for delivery of the acoustic pulses; or
decreasing a power level for delivery of the acoustic pulses.

12. The method of claim 11, wherein:
detecting the ultrasound reflection signals includes use of a plurality of spatially distributed acoustic detectors; and
the level of acoustic activity or the acoustic field distribution is determined at least in part based on locations of the acoustic detectors with respect to the target region.

13. The method of claim 11, wherein the level of acoustic activity or the acoustic field distribution is determined at a plurality of spatially distributed locations.

14. The method of claim 11, wherein:
the method further comprises computing a volumetric acoustic field in the target region by field reconstruction; and
the acoustic pulses have a period in a range of 10 μs to 100 μs.

15. The method of claim 11, further comprising:
estimating the acoustic field distribution for a transmission frequency field, a second harmonic field, a sub harmonic field, and/or an ultra-harmonic field; and
determining a treatment threshold and/or a safety threshold based at least in part on the acoustic field distribution.

16. The method of claim 11, further comprising, based on the plurality of acoustic activity level estimations, increasing the power level for delivery of the acoustic pulses, including increasing the power level at a rate determined at least in part by the acoustic activity level estimations.

17. The method of claim 11, further comprising:
setting the constant power level, the increased power level, or the decreased power level as a working power level for a plurality of points within the target area; and
continuing delivery of the acoustic pulses at the working power level to the plurality of points within the target area without re-estimating the level of acoustic activity or the acoustic field distribution.

18. The method of claim 17, further comprising defining an area including the plurality of points as:
all points at a distance smaller than a predefined radius from an initial point at which the level of acoustic activity or the acoustic field distribution was estimated; or
all points in a segment of the target region having a tissue type corresponding to the tissue type at the initial point at which the level of acoustic activity or the acoustic field distribution was estimated.

19. The method of claim 11, further comprising:
acquiring images using an imaging device comprising a magnetic resonance imaging (MRI) device, a computer tomography (CT) device, or an ultrasound device;
determining a location of an interaction of the acoustic pulses using the images acquired by the imaging device; and
adjusting one or more treatment parameters to shift the location of the interaction to a desired location in the target region.

20. The method of claim 19, wherein:
the images are thermal images of acoustic heating caused by the acoustic pulses, or the images are images sensitive to blood cell extravasation; and
determining the location of the interaction comprises determining the location of the interaction in the images.
